(19) United States
(12) Patent Application Publication
(10) Pub. No.: US 2008/0274509 A1
(43) Pub. Date: Nov. 6, 2008

(54) PROCESS FOR PREPARING SUGAR-CONTAINING HYDROLYZATES FROM LIGNOCELLULOSE

(75) Inventors: Murillo Villela Filho, Bottrop-Kirchhellen (DE); Mirjam Mai, Potsdam (DE); Andreas Karau, Gelnhausen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: EVONIK DEGUSSA GmbH, Essen (DE)

(21) Appl. No.: 12/109,625

(22) Filed: Apr. 25, 2008

(30) Foreign Application Priority Data
Apr. 26, 2007 (DE) 102007019643.3

(51) Int. Cl. C12P 19/00

(52) U.S. Cl. 435/72

(57) ABSTRACT

A sugar-containing hydrolyzate is produced from a lignocellulose-containing material by a) pretreating the lignocellulose-containing material with a chemical compound selected from sulphuric acid, alkali, peroxodisulphates, potassium peroxide, potassium hydroxide, and mixtures thereof, in the presence of water, thereby obtaining an aqueous phase, and b) after removing of the aqueous phase and washing the resulting product, treating said product with an enzyme suitable for hydrolysis in the presence of water, thereby obtaining a hydrolyzate, the hydrolyzate being suitable as a carbon source for fermentation.

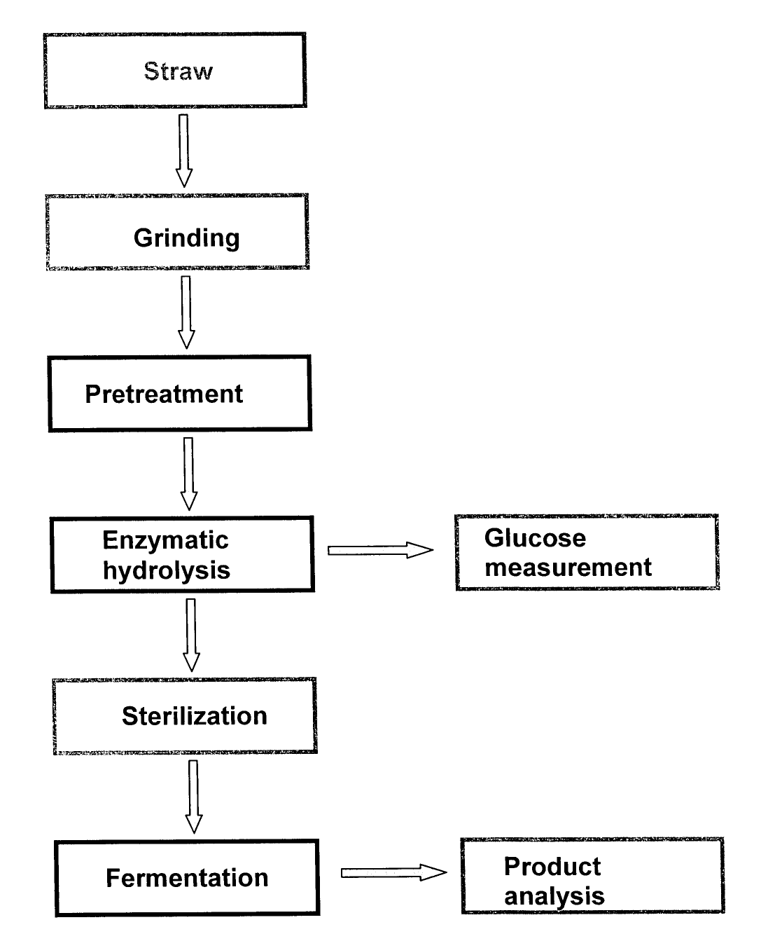

### BACKGROUND OF THE INVENTION

1. Field of the Invention

The invention relates to a solution or suspension which can be used in a fermentation process and is obtained by enzymatic hydrolysis of a lignocellulose-containing material, to a process for its preparation and to its use in the fermentation for preparing organic target substances.

2. Discussion of the Background

Many of the organic target substances are now obtained by biotechnology processes as an alternative to classical processes. In these processes, microorganisms convert the carbon sources supplied to the desired products in fermentation processes. However, the costs of the carbon source restrict the margins in this business. The largest item in the preparation costs of, for example, L-lysine is the carbon source. The price of the sugars most frequently used for this purpose, such as glucose or else sucrose, is subject to high variations and has a major influence on the economic viability of a fermentation process, especially in the case of low-cost mass products such as monosodium glutamate, L-lysine-HCl and L-threonine, the market for which is determined strongly by the competition (M. Ikeda, Advances in Biochemical Engineering/-Biotechnology, Vol. 79 (2003) 2-35).

There are therefore numerous attempts to obtain suitable carbon sources for the fermentation from favourable renewable raw materials.

In addition to starch, cellulose is also formed from glucose monomers and can be split into these monomers by appropriate hydrolysis. For this purpose, different enzymes from those in the starch degradation are needed, since they are β-1,4-linked monomers. Cellulose is a fundamental cell constituent of all plants and thus the most widespread biopolymer worldwide.

Lignocellulose-containing straw is obtained as waste in agriculture and constitutes a less expensive raw material for hydrolyses than starch-containing plants grown specifically for this purpose. However, plant cell walls consist of a network-like mesh of cellulose, hemicellulose and lignin. The latter shield the cellulose from microbial attack, which is important in nature for the stability of the plants. The partly crystalline structure of the cellulose also complicates the enzymatic degradability. This is possible in principle but can be achieved only with poor yields.

For industrial utilization, a high hydrolysis rate with high glucose yield is desired. In addition, the hydrolyzate can be used as a carbon source in fermentations to obtain fine chemicals or ethanol.

An overview of the current state of development in the field of utilization of renewable raw materials in industrial chemical production can be found in R. Busch et al. (Chem. Ing. Tech. 78 (2006) 219-228). According to this, it is possible to convert cellulose and hemicellulose to fermentable products by the action of acids and hydrolytic enzymes.

### Problem

To obtain fermentable hydrolyzates from lignocellulose-containing raw materials, these raw materials, owing to their network-like structure, have to be pretreated physically and chemically before the hydrolysis. Lignocellulose-containing raw materials are understood to mean:
wood from broad-leaved trees and conifers
straw from, for example, maize, rye, wheat, oats, barley, sorghum, rape, rice
bagasse.

In order to be able to provide a larger surface area for the action of chemicals and enzymes, the raw materials are comminuted with mills. Desirable particle sizes are between 0.5-10 mm, preferably between 0.5-5 mm, especially between 1-3 mm.

The chemical pretreatment brings the hemicelluloses present into solution and breaks up the crosslinked aromatic structure of the lignin. The partly crystalline cellulose structure is dissolved and made more accessible for the attack of enzymes.

### BRIEF DESCRIPTION OF DRAWINGS

The FIGURE shows the sequence of the overall process of the present invention.

### DETAILED DESCRIPTION OF THE INVENTION

It is an object of the invention to provide a product which is formed by hydrolysis and is suitable as a carbon source for fermentations to prepare organic target substances.

This and other objects have been achieved by the present invention the first embodiment of which includes a process for preparing a sugar-containing hydrolyzate from a lignocellulose-containing material, comprising:

a) pretreating said lignocellulose-containing material with a chemical compound selected from the group consisting of:

sulphuric acid, alkali, peroxodisulphates, potassium peroxide, potassium hydroxide, and mixtures thereof,

in the presence of water, thereby obtaining an aqueous phase, and

b) after removing of the aqueous phase and washing the resulting product, treating said product with an enzyme suitable for hydrolysis in the presence of water, thereby obtaining a hydrolyzate,

the hydrolyzate being suitable as a carbon source for fermentation.

In another embodiment, the present invention relates to a process for preparing an organic target compound, comprising:

performing the above process, thereby obtaining said sugar-containing hydrolyzate from said lignocellulose-containing material;

preparing said organic target compound by fermentation using at least one microorganism which prepares said organic target compound and by using said sugar-containing hydrolyzate as a carbon source during said fermentation;

wherein said organic target substance has i) at least 3 carbon atoms or ii) at least 2 carbon atoms and one nitrogen atom.

Organic target substances (compounds) include organic substances having at least one nitrogen atom and/or organic substances having at least three carbon atoms and mixtures thereof. Known examples of desired compounds are alcohols, L-amino acids, vitamins, antibiotics, nucleic acids, proteins, enzymes and organic acids. The L-amino acids include especially L-lysine, L-homoserine, L-threonine, L-valine, L-isoleucine, L-proline, L-methionine and L-tryptophan.

The invention provides a process for preparing sugar-containing hydrolyzates from lignocellulose-containing materials, comprising

a) pretreatment of these materials with a chemical compound selected from the group consisting of:

sulphuric acid, alkali, peroxodisulphates, especially potassium peroxodisulphate or ammonium peroxodisulphate, potassium peroxide, potassium hydroxide, in the presence of water, and

b) after removal of the aqueous phase and washing the resulting product, treating it with an enzyme suitable for hydrolysis in the presence of water.

This process uses lignocellulose-containing materials, wood from broad-leaved trees and conifers or straw selected from the plant types of maize, rye, wheat, oats, barley, sorghum, rape or rice, bagasse. The materials can be used alone or in combination.

In order to obtain a larger attack area for the compounds used, the starting materials are comminuted or ground by the known processes.

The term “sugar-containing hydrolyzates” includes contents of the following sugars: hexoses, for example glucose, mannose, galactose and derivatives thereof, for example gluconic acid, glucaric acid or glucuronic acid, or pentoses such as xylose or arabinose and derivatives thereof.

The pH in the pretreatment is between 1 and 14 and is preferably selected depending on the compounds used. The pH includes all values and subvalues therebetween, especially including 2, 3, 4, 5, 6, 7, 8, 9, 10, 11, 12, and 13. The enzymatic hydrolysis is performed generally at a pH in the range of 4 to 7, preferably of 4.5 to 5.5. The pH of the enzymatic hydrolysis includes all values and subvalues therebetween, especially including 4.5, 5, 5.5, 6 and 6.5.

The enzymes used are generally cellulases, e.g. Spezyme Cp from Novozyme (commercially available). Mixtures of enzymes may be used.

For the pretreatment in an acidic medium, dilute sulphuric acid (aqueous sulphuric acid) with a concentration of 0.5-5%, preferably 1-2%, preferably 1-1.5%, is used. % by weight is based on sulphuric acid content (g/100 g). The concentration of the sulphuric acid includes all values and subvalues therebetween, especially including 1, 1.5, 2, 2.5, 3, 3.5, 4 and 4.5% by weight. The acidic straw suspension is stirred in an autoclave with development of autogenous pressure at a temperature of 80-150° C., preferably 90-140° C., preferably 100-130° C., for 90 minutes. The temperature includes all values and subvalues therebetween, especially including 85, 90, 95, 100, 105, 110, 115, 120, 125, 130, 135, 140 and 145° C.

These conditions should be applied correspondingly to the treatment with KOH.

Other known acidic or basic mediums may be used for the pre-treatment.

The oxidizing agents used may be alkali metal peroxodisulphates, especially potassium peroxodisulphate and potassium peroxide, in concentrations of 0.5-5%. The concentration includes all values and subvalues therebetween, especially including 1, 1.5, 2, 2.5, 3, 3.5, 4 and 4.5% by weight. Mixtures of oxidizing agents may be used.

The straw is subsequently removed and preferably washed repeatedly in order to remove the dissolved toxic substances. The washed straw is then used for the hydrolysis and initially charged in the form of a suspension. The enzymes used are conventional such as those of WO 2004/081185. In the subsequent enzymatic hydrolysis, a cellulase complex splits the cellulose chains into smaller fragments down to glucose monomers. The amount of enzyme (protein) should be 0.1-5 g/ml, preferably 0.1-3 g/ml, and should be added in relatively small portions at intervals of a few hours. The amount of enzyme includes all values and subvalues therebetween, especially including 0.5, 1, 1.5, 2, 2.5, 3, 3.5, 4 and 4.5 g/ml. The optimal amount depends on the compound utilized in the pretreatment. The hydrolysis is performed at a temperature of 30-70° C., preferably 40-65°, preferably 50-60° C., over a period of 24 h. The hydrolysis temperature includes all values and subvalues therebetween, especially including 35, 40, 45, 50, 55, 60 and 65° C. The pH should be 3-7, preferably 4-6, preferably 4.5-5.5. The pH includes all values and subvalues therebetween, especially including 3.5, 4, 4.5, 5, 5.5, 6 and 6.5. Subsequently, the solids still present are preferably removed.

After appropriate concentration, which is preferably effected under reduced pressure, the glucose-containing solution is used successfully as a carbon source in fermentations.

The process according to the invention has been demonstrated experimentally by the enzymatic hydrolysis of finely ground maize straw, rye straw and wheat straw (average size 1-3 mm) with a cellulase-enzyme complex (Novozymes, Denmark). The hydrolyzate was concentrated in a vacuum rotary evaporator and then sterilized by autoclaving.

The suitability of wheat and maize straw hydrolyzates prepared in this way as a carbon source for the fermentative preparation of fine chemicals was demonstrated using the example of L-lysine preparation with the Brevibacterium flavum DM1730 strain (Georgi et al. 2005) in shaken-flask experiments with monitoring of the metabolic activity. In addition, it was found that, surprisingly, some of the experiments with hydrolyzate led to significantly higher product formation than the control experiment with standard glucose solution.

The invention therefore likewise provides a process for preparing an organic target compound, comprising:

performing the above described process, thereby obtaining a sugar-containing hydrolyzate from the lignocellulose-containing material;

preparing an organic target compound by fermentation using at least one microorganism which prepares said organic target compound and by using said sugar-containing hydrolyzate as a carbon source during said fermentation;

wherein said organic target substance has i) at least 3 carbon atoms or ii) at least 2 carbon atoms and one nitrogen atom.

This can optionally be supplemented by further carbon sources.

After the fermentation, the target substance is preferably isolated, optionally with the total amount or parts of the biomass. The fermentation broth is generally concentrated in a gentle manner beforehand, i.e. without decomposition of constituents. In the FIGURE, the sequence of the overall process is reproduced.

In the process according to the invention, especially target products are prepared which are selected from the group of: mono-, di- and tricarboxylic acids having 3 to 10 carbon atoms, proteinogenic and non-proteinogenic L-amino acids, saturated and unsaturated fatty acids; diols having 3 to 8 carbon atoms, polyhydric alcohols having 3 or more hydroxyl groups, relatively long-chain alcohols having at least 4 carbon atoms, vitamins, provitamins, ketones having 3 to 10 carbon atoms.

The target products are selected especially from the group consisting of L-amino acids and vitamins, especially L-lysine, L-methionine, L-threonine, L-proline, L-isoleucine, L-homoserine, L-valine, pantothenic acid and riboflavin, and also propionic acid, propanediol, butanol and acetone.

The microorganisms are selected from those which produce especially L-amino acids or vitamins, especially from microorganisms which produce L-lysine, L-methionine, L-threonine, L-proline, L-isoleucine, L-homoserine, L-valine, pantothenic acid, riboflavin, propionic acid, propanediol, butanol, acetone and trehalose.

Producing microorganisms are understood to mean those which produce the desired target products to an increased degree compared to the wild type and may excrete them.

The fermentation of the microorganisms can be performed continuously—as described, for example, in PCT/EP 2004/008882—or batchwise in a batch process (batch cultivation) or in a fed batch process or repeated fed batch process to produce the target substances. A general review of known cultivation methods is available in the textbook by Chmiel (Bioprozesstechnik 1. Einführung in die Bioverfahrenstechnik [Bioprocess technology 1. Introduction into bioprocess technology] (Gustav Fischer Verlag, Stuttgart, 1991)) or in the textbook by Storhas (Bioreaktoren und periphere Einrichtungen [Bioreactors and peripheral devices] (Vieweg Verlag, Braunschweig/Wiesbaden, 1994)).

The culture medium or fermentation medium to be used has to satisfy the demands of the particular strains in a suitable manner. Descriptions of culture media of different microorganisms are present in the handbook “Manual of Methods for General Bacteriology” of the American Society for Bacteriology (Washington D.C., USA, 1981). The terms “culture medium” and “fermentation medium” or “medium” are interchangeable.

The carbon sources used may, as well as sugars obtained by hydrolysis, be further sugars and carbohydrates, for example glucose, sucrose, lactose, fructose, maltose, molasses, sucrose-containing solutions from sugarbeet or sugarcane production, starch, starch hydrolyzate and cellulose, oils and fats, for example soya oil, sunflower oil, groundnut oil and coconut fat, fatty acids, for example palmitic acid, stearic acid and linoleic acid, alcohols, for example glycerol, methanol and ethanol, and organic acids, for example acetic acid. These substances may be used individually or as a mixture.

The nitrogen sources used may be organic nitrogen compounds such as peptones, yeast extract, meat extract, malt extract, corn steep liquor, soybean meal and urea, or inorganic compounds such as ammonia, ammonium sulphate, ammonium phosphate, ammonium carbonate and ammonium nitrate, preferably ammonia or ammonium sulphate. The nitrogen sources may be used individually or as a mixture.

The phosphorus sources used may be phosphoric acid, potassium dihydrogenphosphate or dipotassium hydrogenphosphate, or the corresponding sodium salts.

The culture medium must additionally contain salts, for example in the form of sulphates of metals, for example sodium, potassium, magnesium, calcium and iron, for example magnesium sulphate or iron sulphate, which are needed for growth. Finally, essential growth factors such as amino acids, for example homoserine, and vitamins, for example thiamine, biotin or pantothenic acid, may be used in addition to the above-mentioned substances. Moreover, suitable precursors of the particular amino acid can be added to the culture medium.

The feedstocks used are added to the culture in the form of a single mixture or fed in during the cultivation in a suitable manner.

For pH control of the culture, basic compounds such as sodium hydroxide, potassium hydroxide, ammonia or aqueous ammonia, preferably ammonia or aqueous ammonia, or acidic compounds such as phosphoric acid or sulphuric acid, are used in a suitable manner. The pH is generally adjusted to a value of 6.0 to 9.0, preferably 6.5 to 8. The pH includes all values and subvalues therebetween, especially including 6.5, 7, 7.5, 8 and 8.5. To control the evolution of foam, it is possible to use antifoams, for example fatty acid polyglycol esters. To maintain the stability of plasmids, suitable selective substances, for example antibiotics, can be added to the medium. In order to maintain aerobic conditions, oxygen or oxygenous gas mixtures, for example air, are introduced into the culture. The use of liquids enriched with hydrogen peroxide is likewise possible. If appropriate, the fermentation is conducted at elevated pressure, for example at a pressure of 0.03 to 0.2 MPa. The pressure includes all values and subvalues therebetween, especially including 0.04, 0.05, 0.06, 0.08, 0.1, 0.12, 0.14, 0.16 and 0.18. The temperature of the culture is normally 20° C. to 45° C. and preferably 25° C. to 40° C. The temperature of the culture includes all values and subvalues therebetween, especially including 25, 30 and 35° C. In batch processes, the cultivation is continued until a maximum of the desired amino acid has formed. This aim is normally achieved within 10 hours to 160 hours. The time includes all values and subvalues therebetween, especially including 20, 30, 40, 50, 60, 70, 80, 90, 100, 110, 120, 130, 140 and 150 hours. In continuous processes, longer cultivation times are possible.

Examples of suitable fermentation media can be found, inter alia, in the patents U.S. Pat. No. 5,770,409, U.S. Pat. No. 5,840,551 and U.S. Pat. No. 5,990,350, U.S. Pat. No. 5,275,940 or U.S. Pat. No. 4,275,157. Further examples of fermentation media can be found in Ozaki and Shiio (Agricultural and Biological Chemistry 47(7), 1569-1576, 1983) and Shiio et al. (Agricultural and Biological Chemistry 48(6), 1551-1558, 1984).

In a preferred embodiment, the product prepared by fermentation is an L-amino acid, especially L-lysine. To perform the fermentation, it is possible here to employ analogous conditions and procedures, as described, for example, in Pfefferle et al., Advances in Biochemical Engineering/Biotechnology, Vol. 79 (2003) 60-112 and in U.S. Pat. No. 3,708,395.

Microorganisms used in accordance with the invention are preferably selected from the genera Corynebacterium, Bacillus, Escherichia, Aspergillus, Lactobacillus, especially from strains of Corynebacterium glutamicum, Bacillus subtilis, Escherichia coli or Aspergillus niger.

Many chemical methods for pretreatment of cellulose to enhance the hydrolyzates are known. However, not all hydrolyzates are suitable as a carbon source for fermentation. A particularly suitable method is the inventive pretreatment with sulphuric acid or potassium hydroxide and some selected oxidizing agents, for example potassium peroxide or potassium peroxodisulphate.

It has likewise been shown that this is not the case for the alkaline pretreatment with sodium hydroxide (performed according to Varga et al.) Varga et al., Applied Biochemistry and Biotechnology, vol. 98-100 (2002) 75-86, and for the oxidative pretreatments with peracetic acid, hydrogen peroxide or sodium hypochlorite (performed according to WO 2004/081185), and the combined use of sodium hydroxide and sulphuric acid (2-step, according to Varga et al.). Toxic by-products possibly form, which prevent the growth of microorganisms, such that no product formation takes place either.

Having generally described this invention, a further understanding can be obtained by reference to certain specific examples which are provided herein for purposes of illustration only, and are not intended to be limiting unless otherwise specified.

### EXAMPLES

It has been shown that only selected pretreatment methods, such as the use of sulphuric acid or particular oxidizing agents, lead to hydrolyzates which can be used as the carbon source in fermentation processes.

1. Pre-Treatment of the Present Invention

1.1 Sulphuric Acid

Concentrated sulphuric acid (oleum, 96%) was used to prepare a 1.2% solution by dilution. 10 g of ground straw were filled into a tantalum autoclave and admixed with 500 ml of the dilute acid. With development of autogenous pressure, the autoclave was heated to 120° C. At this temperature, the straw suspension was stirred for 90 min and then cooled. The operation was repeated, such that 1 l of the pretreated straw suspension was available for the hydrolysis.

1.2 Oxidizing Agent

20 g of straw were suspended in about 800 ml of demineralized water. The oxidizing agent was dissolved in about 200 ml of demineralized water and added slowly to the straw suspension by means of a dropping funnel. Details of the exact amounts can be taken from Table 1. The rate of dropwise addition should be about 1 drop per minute in order to prevent an excessive temperature rise. As soon as the addition had ended, the thermostat was switched on and the suspension was heated to 80° C. With stirring (150 rpm), the suspension was kept at this temperature for 24 h. Subsequently, the presence of the oxidizing agent was tested with peroxide test sticks or potassium iodide-starch paper, and optionally destroyed with catalytic amounts of iron(II) sulphate.

TABLE 1
Amounts of oxidizing agent used

| | KO₂ | K₂S₂O₈ | KOH |
|---|---|---|---|
| Demineralized water for | 800 ml | 800 ml | 1000 ml |
| suspension | | | |
| Oxidizing agent | 5 g | 10 g | 4 g |
| Demineralized water for | 200 ml | 200 ml | — |
| dissolution | | | |

2. Comparative Treatment

2.1 Alkaline (According to Varga et al.)

NaOH pellets were used to prepare 1 l of a 1% solution. 10 g of ground straw were filled into a 1 l Hastelloy autoclave and admixed with 500 ml of the dilute sodium hydroxide solution. With development of autogenous pressure, the autoclave was heated to 120° C. At this temperature, the straw suspension was stirred for 60 min and then cooled. The operation was repeated, such that 1 l of the pretreated straw suspension was available for the hydrolysis.

2.2 2-Step (According to Varga et al.)

20 g of straw were suspended at room temperature in 1% sodium hydroxide solution with stirring (150 rpm) for 24 h. Subsequently, the straw was filtered off with suction through a filter by means of a vacuum pump and washed repeatedly with demineralized water. The straw was suspended in 1% sulphuric acid in an autoclave and stirred at 120° C. with development of autogenous pressure for 1 h.

3. Oxidizing Agents (According to WO 2004/081185)

As under inventive pretreatment, but different oxidizing agents (Tab. 2)

TABLE 2
Amounts of oxidizing agent used

| | NaOCl | CH₃COOOH | H₂O₂ | |
|---|---|---|---|---|
| Demineralized water | 750 ml | 800 ml | — | |
| for suspension | | | | |
| Oxidizing agent | 100 ml | 26 ml | 6 | ml |
| Demineralized water | 150 ml | 174 ml | 994 | ml |
| for dissolution | | | | |

4. Enzymatic Hydrolysis

The straw suspension pretreated as described under 1 and 2 was filled into a 6 l fermentor and adjusted to pH 5.0 with 10 mM citrate buffer and 10 M sodium hydroxide solution or concentrated sulphuric acid. The suspension was stirred at 100 rpm and heated to 60° C., and then 2 ml (0.133 g/ml of protein) of Spezyme Cp enzyme preparation (Novozymes, Denmark) were added. The hydrolysis was monitored over 24 h by regular measurements of the glucose concentration. Thereafter, the hydrolysis was ended by cooling.

5. Fermentation

5.1 Standard

A CgXII standard medium comprising pure glucose was prepared by the recipe of Keilhauer et al. (J. Bacteriol., Vol. 175 (1993) 5595) with a reduced glucose content (see Table 3) and sterilized in an autoclave. The nitrogen-containing salts were dissolved separately in demineralized water and sterilized in an autoclave. Biotin and protocatechuic acid stock solution were not thermally sterilized but made up freshly and added to the medium through a sterile filter (pore size 0.2 μm).

5.2 Hydrolyzates

The hydrolyzates were used to prepare the media according to the recipe of Keilhauer et al. for CgXII medium. The carbon sources used were the concentrated hydrolyzates in which the nutrient salts were dissolved. For comparison, a standard medium comprising pure glucose was prepared. The glucose concentration in all media was adjusted such that all have the same concentration before the sterilization. After the sterilization, they may, however, differ.

TABLE 3
CgXII medium (according to Keilhauer et al. 1993, modified)

| | | | | | Stock | |
|---|---|---|---|---|---|---|
| | End | | | | solution | |
| | concen- | | Volume | | withdrawn | |
| Substance | tration | | [ml] | Weight | [ml] | pH |
| (NH₄)₂SO₄ | 20 | g/l | 150 | 3 g | 0.15 | 7 |
| Urea | 5 | g/l | 150 | 0.75 | 0.15 | 7 |
| KH₂PO₄ | 1 | g/l | 150 | 0.15 | 0.15 | 7 |
| K₂HPO₄ | 1 | g/l | 150 | 0.15 | 0.15 | 7 |
| MgSO₄ × 7H₂O | 0.25 | g/l | 150 | 0.0375 | 0.15 | 7 |
| MOPS | 42 | g/l | 150 | 6.3 | 0.15 | 7 |
| CaCl₂ | 10 | mg/l | 20 | 0.2 ml | 0.15 | 7 |
| FeSO₄ × 7H₂O | 10 | mg/l | 50 | 0.5 | 0.15 | 1 |
| MnSO₄ × 1H₂O | 10 | mg/l | 50 | 0.5 | 0.15 | 1 |
| ZnSO₄ × 7H₂O | 1 | mg/l | 50 | 0.05 | 0.15 | 1 |
| CuSO₄ × 5H₂O | 0.2 | mg/l | 50 | 0.0155 | 0.15 | 1 |
| NiCl₂ × 6H₂O | 0.02 | mg/l | 50 | 0.001 | 0.15 | 1 |
| Biotin | 0.2 | mg/l | 20 | 0.004 | 0.15 | — |
| Protocatechuic acid | 30 | mg/l | 20 | 0.06 | 1.5 | 6.5 |
| Glucose | 16 | g/l | 200 | 103.2 | 4.651 | — |

5.3 Preculture

A sterile sealable test tube was filled with 2 ml of standard medium, inoculated with a bacteria culture of the Brevibacterium flavum strain DM1730 (Georgi et al. VF (2005) Metab. Eng. A(4): 291-301) from a plate and incubated at 31° C. in a heated cabinet. After 24 h, this preculture was used to inoculate the shaken flask. To this end, the OD (optical density) of the preculture was determined at a wavelength of 600 nm and then diluted with sterile water to the desired OD.

5.4 Main Fermentation

To determine the metabolic activity, the fermentation is effected with the RAMOS system (from HiTec Zang, Herzogenrath). This imitates a bioprocess in a conventional shaken flask. It records the data obtained from the partial pressure measurements for the oxygen transfer rate (OTR), the carbon dioxide transfer rate (CTR) and the respiration quotient (RQ), and plots them graphically in the attached computer unit.

The liquid volume of the Ramos flask must not exceed 10 ml, and 1% of the volume is estimated for inoculation. This gives rise to an inoculum of 100 μl which is added to 9.9 ml of medium. The flasks are filled, weighed and secured in the shaker. After testing for leaks and oxygen calibration, the measurement is started. With reference to the data recorded by the computer, the profile of the metabolic activity is monitored. The measurement is always effected as a double determination. After the fermentation has ended, OD, glucose content and amino acid content are determined.

The OTR curves of the standard medium and of three hydrolysates (AK sulphuric acid, AK sodium hydroxide solution, potassium peroxide) were recorded. The standard medium contains pure glucose and serves for comparison of the curve profile. After about 12 h, the glucose in the standard medium has been consumed and the bacteria die off.

The bacteria in the sulphuric acid medium grow significantly later than those in the standard medium. An inhibitor appears to be present, which is responsible for the longer lag phase (adaptation phase). The bacteria first have to adapt their metabolism to these conditions before they enter the growth phase. Their growth rate there is, however, just as high as that in the standard medium. These inhibitors might be furfural and 5-hydroxymethylfurfural which are formed in the course of acid treatment of sugars. They are known as fermentation inhibitors. After 15 h, a shoulder can be seen in the curve. This indicates diauxic growth. Here, the bacterium switches its metabolism to further carbon sources; as a result, the metabolic activity initially remains constant.

The medium from the oxidative pretreatment allows equally early growth to that in the standard medium and also the same growth rate. After 10 h, a shoulder can likewise be seen in the curve profile. A second occurs after 14 h. This means that C. glutamicum, apart from glucose, metabolizes to other carbon sources which are present in the hydrolysate. As well as other sugars from the hemicellulose, they may also be sugar derivatives. One supposition is gluconic acid, which is formed in the oxidation of glucose by hydrolysis of initially formed glucono-o-lactone. Gluconic acid is an intermediate of the metabolism of C. glutamicum and might therefore be discharged from the medium into the metabolic pathway.

Acetate which is released from the hemicellulose can be cometabolized by C. glutamicum. This means that it is utilized simultaneously with glucose without the metabolism being switched. Acetate thus does not bring about any diauxie. The citrate from the hydrolysis buffer can likewise be utilized by C. glutamicum. In the hydrolysate of the pretreatment with NaOH, one substance appears to be toxic for the bacteria, since the metabolic activity is completely inhibited.

5.5 Preparation of L-Lysine

Table 4 lists the results of the fermentation which were found using the hydrolyzates prepared in accordance with the invention.

TABLE 4

| | pH of the | Glucose after | Glucose before | Lysine after |
|---|---|---|---|---|
| Pretreatment with | pretreatment | hydrolysis [g/l] | fermentation [g/l] | fermentation [g/l] |
| Sulphuric acid* | 1 | 2.93 | 9.6 | 4.68 |
| Potassium peroxide* | 10-11 | 3.42 | 9.0 | 4.47 |
| Potassium peroxodisulphate | 1-2 | 4.7 | 11.12 | 6.71 |
| Potassium hydroxide | 10 | 12.1 | 16.92 | 6.71 |

Further materials: *20 g of maize straw, 2 ml of enzyme
Others: 61 g of wheat straw, 40 ml of enzyme

Table 5 contains the results achieved using hydrolyzates not prepared in accordance with the invention. It was found that, even though the fermentation broth contains glucose, it had not metabolized for the preparation of L-lysine.

This fact demonstrates that particular conditions have to be maintained to prepare utilizable hydrolyzates.

TABLE 5

| | pH of the | Glucose after | Glucose before | Lysine after |
|---|---|---|---|---|
| Pretreatment with | pretreatment | hydrolysis [g/l] | fermentation [g/l] | fermentation |
| Peracetic acid* | 2-3 | 2.35 | 9.0 | 0 |
| Sodium hydroxide* | 14 | 2.52 | 8.95 | 0 |
| Hydrogen peroxide* | 3 | 6.66 | 13.28 | 0 |
| Sodium hypochlorite | 7 | 2.56 | 11.76 | 0 |
| 2-Step* | First 10, then 1 | 4.07 | 14.84 | 0 |

Further materials: *= 20 g of maize straw, 2 ml of enzyme;
Others: 61 g of wheat straw, 40 ml of enzyme

German patent application 10 2007 019 643.3 filed Apr. 26, 2007, is incorporated herein by reference.

Numerous modifications and variations on the present invention are possible in light of the above teachings. It is therefore to be understood that within the scope of the appended claims, the invention may be practiced otherwise than as specifically described herein.

1. A process for preparing a sugar-containing hydrolyzate from a lignocellulose-containing material, comprising:
a) pretreating said lignocellulose-containing material with a chemical compound selected from the group consisting of:
sulphuric acid, alkali, peroxodisulphates, potassium peroxide, potassium hydroxide, and mixtures thereof,
in the presence of water, thereby obtaining an aqueous phase, and
b) after removing of the aqueous phase and washing the resulting product, treating said product with an enzyme suitable for hydrolysis in the presence of water, thereby obtaining a hydrolyzate,
the hydrolyzate being suitable as a carbon source for fermentation.

2. The process according to claim 1, wherein the lignocellulose-containing material is wood from broad-leaved trees and conifers.

3. The process according to claim 1, wherein the lignocellulose-containing material is straw of a plant selected from the group consisting of maize, rye, wheat, oats, barley, sorghum, rape, rice, bagasse and combinations thereof.

4. The process according to claim 1, wherein sulphuric acid or alkali metal peroxodisulphates are used at a pH of 1 to 3.

5. The process according to claim 1, wherein potassium peroxide or potassium hydroxide is used at a pH of 9 to 12.

6. A process for preparing an organic target compound, comprising:
performing the process according to claim 1, thereby obtaining said sugar-containing hydrolyzate from said lignocellulose-containing material;
preparing said organic target compound by fermentation using at least one microorganism which prepares said organic target compound and by using said sugar-containing hydrolyzate as a carbon source during said fermentation;
wherein said organic target substance has i) at least 3 carbon atoms or ii) at least 2 carbon atoms and one nitrogen atom.

7. The process according to claim 6, in which, on completion of the fermentation, the target compound is isolated, optionally together with a total amount or a part of a biomass formed during the fermentation.

8. The process according to claim 6, wherein said organic target compound is selected from the group consisting of organic, optionally hydroxyl-bearing mono-, di- and tricarboxylic acids having 3 to 10 carbon atoms, proteinogenic and non-proteinogenic L-amino acids, saturated and unsaturated fatty acids, diols having 3 to 8 carbon atoms, polyhydric alcohols having 3 or more hydroxyl groups, long-chain alcohols having at least 4 carbon atoms, vitamins, provitamins, ketones having 3 to 10 carbon atoms and combinations thereof.

9. The process according to claim 6, wherein said organic target compound is selected from the group consisting of L-amino acids, vitamins, propionic acid, propanediol, butanol, acetone, trehalose and mixtures thereof.

10. The process according to claim 6, wherein said microorganism is selected from the group consisting of microorganisms which overproduce L-amino acids, vitamins, propionic acid, propanediol, butanol, acetone, trehalose and mixtures thereof.

11. The process according to claim 6, wherein said microorganism is selected from the group consisting of the genera Corynebacterium, Bacillus, Escherichia, Aspergillus, Lactobacillus, Clostridium and combinations thereof.

12. The process according to claim 6, wherein said organic target compound is selected from the group consisting of L-lysine, L-methionine, L-threonine, L-proline, L-isoleucine, L-homoserine, L-valine and mixtures thereof.

13. The process according to claim 6, wherein said organic target compound is selected from the group consisting of pantothenic acid, riboflavin and mixtures thereof.

14. The process according to claim 6, wherein said microorganism is selected from the group consisting of microorganisms which overproduce L-lysine, L-methionine, L-threonine, L-proline, L-isoleucine, L-homoserine, L-valine and mixtures thereof.

15. The process according to claim 6, wherein said microorganism is selected from the group consisting of microorganisms which overproduce pantothenic acid, riboflavin and mixtures thereof.

16. The process according to claim 6, wherein said microorganism is selected from the group consisting of the Corynebacterium glutamicum, Bacillus subtilis, Escherichia coli, Aspergillus niger and combinations thereof.

17. The process according to claim 1, wherein said hydrolysis is performed at a temperature of 30-70° C.

18. The process according to claim 1, wherein said lignocellulose-containing material is pretreated in aqueous sulphuric acid at a temperature of 80-150° C.

19. The process according to claim 1, wherein potassium peroxodisulphate or ammonium peroxodisulphate are used for the pre-treatment.

20. The process according to claim 1, wherein alkali metal peroxodisulphate is used for the pre-treatment in a concentration of 0.5-5% by weight based on the weight of the aqueous phase.
